(19) United States
(12) Patent Application Publication
(10) Pub. No.: US 2022/0189627 A1
(43) Pub. Date: Jun. 16, 2022

(54) DRIVE-THROUGH MEDICAL TREATMENT SYSTEM AND DRIVE-THROUGH MEDICAL TREATMENT METHOD

(71) Applicants: HYUNDAI MOTOR COMPANY, SEOUL (KR); KIA CORPORATION, SEOUL (KR)

(72) Inventor: Joon Young Kim, Seongnam-si (KR)

(21) Appl. No.: 17/383,112

(22) Filed: Jul. 22, 2021

(30) Foreign Application Priority Data
Dec. 11, 2020 (KR) 10-2020-0173319

(51) Int. Cl. G16H 40/67; H04B 17/318; H04W 4/021

(52) U.S. Cl. CPC G16H 40/67; H04W 4/021; H04B 17/318

(57) ABSTRACT

A drive-through medical treatment system utilizing a vehicle, includes a plurality of medical treatment apparatuses provided in a plurality of medical shelters, and a central management apparatus configured to receive information on a medical treatment result and information on service user from the plurality of medical treatment apparatuses, to identify a progress situation of the drive-through medical treatment, and to send required information and a control instruction to each of the plurality of medical treatment apparatuses such that medical treatment required for service user may be performed based on identified situation, where at least one of the plurality of medical treatment apparatuses is configured to receive information on the service user to generate medical treatment data, to process information on a medical treatment result of the service user, and to transmit the processed information to an adjacent medical treatment apparatus and the central management apparatus.

### CROSS REFERENCE TO RELATED APPLICATIONS

This application claims priority to and the benefit of Korean Patent Application No. 10-2020-0173319 filed in the Korean Intellectual Property Office on Dec. 11, 2020, the entire contents of which are incorporated herein by reference.

### BACKGROUND

### (a) Field

The present disclosure relates to a drive-through medical treatment system and a drive-through medical treatment method.

### (b) Description of the Related Art

In an existing drive-through medical examination service, a delivery of a mere information and a limited level of medical examination is provided. Even if information of a service user is delivered to the medical staff, only the information directly written by the service user is delivered to the medical staff due to environmental constraints of the medical examination service. In many cases, the amount of information is not sufficient because the service user may feed inconvenient to write such information. This delivery of such limited information increases the time required to provide medical examination services.

The above information disclosed in this Background section is only for enhancement of understanding of the background of the disclosure, and therefore it may contain information that does not form the prior art that is already known in this country to a person of ordinary skill in the art.

### SUMMARY

The present disclosure has been made in an effort to provide a drive-through medical treatment system and method capable of reducing time required for providing medical examination service.

A drive-through medical treatment system utilizing a vehicle, may include a plurality of medical treatment apparatuses provided in a plurality of medical shelters, and a central management apparatus to receive information on a medical treatment result and information on service user from the plurality of medical treatment apparatuses, to identify a progress situation of the drive-through medical treatment, and to send required information and a control instruction to each of the plurality of medical treatment apparatuses such that medical treatment required for service user may be performed based on identified situation, where at least one of the plurality of medical treatment apparatuses may be configured to receive information on the service user to generate medical treatment data, to process information on a medical treatment result of the service user, and to transmit the processed information to an adjacent medical treatment apparatus and the central management apparatus.

The vehicle may include a sensor device configured to check a current status of the service user, a data processing/formatting device configured to process information on the service user obtained through the sensor device according to data format, to generate service user information, and a communication unit configured to transmit the service user information to the plurality of medical treatment apparatuses.

The information on the service user may include an image photographing a face of the service user, a body temperature and heart rate of the service user during a predetermined monitoring period, and voice data of the service user.

The data processing/formatting device may be configured to analyze the information on the service user, to extract a maximum heart rate of the service user during a predetermined monitoring period, to extract a maximum level of voice of the service user, to extract a maximum temperature from measured body temperatures of the service user, and to estimate at least one of a health status and an emotional status of the service user based on a photographed image of the service user, and may be configured to process the analyzed information in a text data format to generate the service user information.

Each of the plurality of medical treatment apparatuses may include a controller configured to monitor operations performed by each medical treatment apparatus and to perform a control operation according to a monitoring result, a first communication unit performing communication with the vehicle, a data gateway configured to receive data from the first communication unit or to transmit data to the first communication unit, an analysis/format device configured to analyze data received from the data gateway to select a data format, and a data processor configured to process the data received from the data gateway according to the data format selected by the analysis/format device to generate medical treatment data.

The drive-through medical treatment system may further include a module processor configured to receive the medical treatment data from the data processor, and to perform processing of information required for performing medical treatment based on the medical treatment data.

The analysis/format device may be configured to select a protocol and format that may be used for data processing from a protocol and format table based on a result of analyzing the received data, and to inform the selected data format to the data processor.

The analysis/format device may be configured to, when a protocol and format that may be used for data processing does not exists in a protocol and format table, to the controller that the protocol and format suitable for the data received from the vehicle does not exist. The controller may be configured to request the information on protocol and format from the central management apparatus.

The controller may be configured to receive information on changed protocol and format from the central management apparatus, and to deliver the received information to the analysis/format device. The analysis/format device may be configured to add the delivered protocol and format to the protocol and format table, and to select the added data format to transmit the selected data format to the data processor.

Each of the plurality of medical treatment apparatuses may further include a distance determiner configured to measure a strength of a signal received from the vehicle, and to estimate a distance between the vehicle and a medical shelter based on the measured received signal strength indication (RSSI).

The drive-through medical treatment system may further include a status mode device configured to, when an Enable signal is generated due to determining by the distance determiner that the vehicle has entered a corresponding medical shelter, enable the data gateway and the module processor synchronously with the Enable signal.

The central management apparatus may include a client configured to collect information on protocol and format from the plurality of medical treatment apparatuses, and to transmit information on updated protocol and format to the plurality of medical treatment apparatuses, and an analysis determiner configured to analyze the information on protocol and format collected by the client to calculate a usage rate and a data transmission success rate with respect to each pair within the protocol and format group, and to set ranking for each pair according to the usage rate and the data transmission success rate.

The analysis determiner may be configured to, when each of the usage rate and the success rate is below a predetermined threshold value, or when a sum value of values obtained by multiplying each of the usage rate and the success rate by a reflection ratio is below a predetermined threshold value, decide to discard a corresponding pair within the protocol and format group decision.

The central management apparatus may further include a reconstruction device configured to generate a new protocol and format pair when a discarded pair exists in the protocol and format group.

The central management apparatus may further include a remapping device performing remapping with respect to the protocol and format group by excluding the protocol and format pair discarded by the analysis determiner, and by including the new protocol and format pair generated by the reconstruction device.

A drive-through medical treatment method for receiving a drive-through medical treatment service by using a vehicle, may include detecting, by a distance determiner, a signal from the vehicle, collecting, by the distance determiner, signals received from the vehicle, and calculating an average of strength of the received signal, determining, by the distance determiner, whether the calculated average of received signal strength is larger than a predetermined reference value, enabling, by the distance determiner, a status mode device, when the average of the received signal strength is larger than the reference value, enabling, by the status mode device, a data gateway to be turned on, determining, by the turned-on data gateway, whether the data received from the vehicle has an essential credential, and analyzing, by an analysis/format device, the data delivered from the data gateway to select a corresponding data format in a protocol and format table, when the received data has the essential credential.

The drive-through medical treatment method may further include, repeating, by the status mode device, an enable operation with respect to the data gateway by a predetermined number of times repeat, when the data gateway is not enabled by the status mode device.

The drive-through medical treatment method may further include, requesting the vehicle, by the data gateway, to collect data required for the credential, when the essential credential does not exist in the data received from the vehicle, and repeating, by the data gateway, data collect by a predetermined number of times, when a response with respect to the collection request is not received.

A drive-through medical treatment method, may include, analyzing, by an analysis/format device, data received from a vehicle, determining, by the analysis/format device, a suitable data format for the analyzed data, based on a protocol and format table, reconstructing, when the suitable data format does not exists, by the analysis/format device, the protocol and format table according to a new protocol and format received from central management apparatus, and transmitting the new data format to a data processor, and reconstructing, by the data processor, the data received from the vehicle according to the data format.

A drive-through medical treatment method, may include, detecting, by the drive-through medical treatment system, by using a geo-fence, a vehicle entering a drive-through medical treatment service area, informing a plurality of medical treatment apparatuses, by the drive-through medical treatment system, when the vehicle enters the geo-fence, detecting, by a distance determiner of at least one of the plurality of medical treatment apparatuses, the vehicle, and guiding the vehicle to move to a first medical shelter in a preset medical treatment sequence, transmitting information on a service user to the medical treatment apparatus through communication between the vehicle and the medical treatment apparatus of the first medical shelter, processing, by the medical treatment apparatus, received data according to a data format to generate medical treatment data, and processing an inputted medical treatment result according to the data format to generate medical treatment data after completing a medical examination, and transmitting the generated medical treatment data to a medical treatment apparatus of a subsequent shelter among the plurality of medical treatment apparatuses.

According to an exemplary embodiment of the present disclosure, a drive-through medical treatment system and method that is capable of reducing time required for providing a medical examination service is provided.

### BRIEF DESCRIPTION OF THE FIGURES

FIG. 1 illustrates a drive-through medical treatment system according to an exemplary embodiment.

FIG. 2 illustrates an exemplary configuration of a vehicle and a medical treatment apparatus according to an exemplary embodiment.

FIG. 3 illustrates determining protocol and format according to an exemplary embodiment.

FIG. 4 illustrates an example of reconstructing a data format according to an exemplary embodiment.

FIG. 5 is a flowchart showing a drive-through medical treatment method according to an exemplary embodiment.

FIG. 6 illustrates a data format determining method according to an exemplary embodiment.

FIG. 7 illustrates an example applied with a drive-through medical treatment system according to an exemplary embodiment.

FIG. 8 illustrates another example applied with a drive-through medical treatment system according to an exemplary embodiment.

### DETAILED DESCRIPTION

The present disclosure may perform collecting of various information and medical examination/medical treatment based on communication/network and platform conjunction construction. By selecting the optimal data format for service user information and applying various vehicle communication/data techniques to service user information delivery, it is possible to secure flexibility of information delivery and minimize data transmission.

The present disclosure will be described more fully hereinafter with reference to the accompanying drawings, in which exemplary embodiments of the disclosure are shown. As those skilled in the art would realize, the described embodiments may be modified in various different ways, all without departing from the spirit or scope of the present disclosure.

An embodiment of the present disclosure is hereinafter described in detail with reference to the drawings.

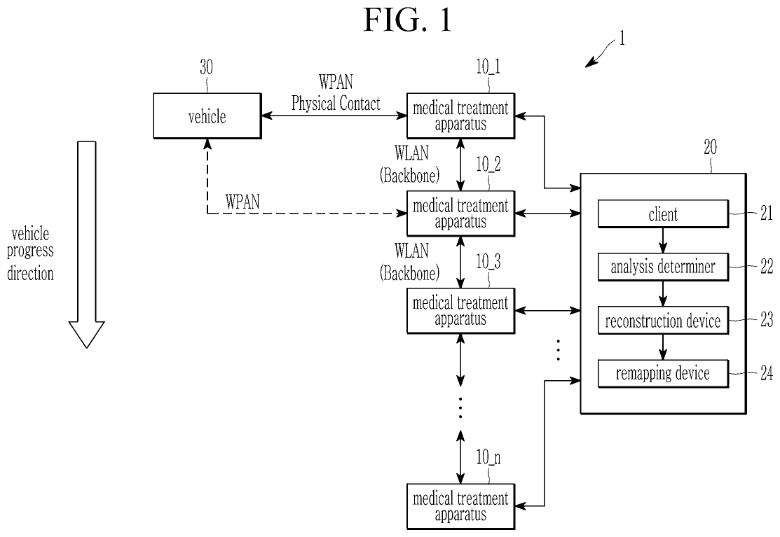

FIG. 1 illustrates a drive-through medical treatment system according to an exemplary embodiment.

A drive-through medical treatment system 1 may be applied to a drive-through medical treatment place, where a plurality of medical shelters are positioned, and a person subject to medical treatment riding on a vehicle receives medical treatment while passing through a plurality of medical shelters by the vehicle. In each of the plurality of medical shelters, inspection, medical treatment, or an event (such as reception, reservation, etc.) related to any one of inspection and medical treatment is performed, and such medical shelters are provided with apparatuses appropriate for the work of the medical shelters. Hereinafter, an equipment provided to the medical shelter is called a medical treatment apparatus. When medical treatment is performed in a medical shelter, a doctor may be positioned in the corresponding medical shelter, and when inspection is performed in the medical shelter, a medial specialist or engineer for inspection may be positioned.

The drive-through medical treatment system 1 includes a plurality of medical treatment apparatuses 10_1 to 10_n, and a central management apparatus 20.

As shown in FIG. 1, between nearby medical treatment apparatus among the plurality of medical treatment apparatuses 10_1 to 10_n, required information is sent and received through a wireless local area network (WLAN), and between the vehicle entering the drive-through medical treatment system 1 and the plurality of medical treatment apparatuses 10_1 to 10_n, required information is sent and received through a wireless personal area network (WPAN).

A service user who uses drive-through medical treatment service is riding in a vehicle 30. When the vehicle 30 cannot communicate with the plurality of medical treatment apparatuses 10_1 to 10_n, a personal terminal used by the service user instead of the vehicle 30 may be applied with a drive-through medical treatment service.

Each of the plurality of medical treatment apparatuses 10_1 to 10_n includes various configurations necessary to perform medical treatment to the service user for at least one specific subject. Each of the plurality of medical treatment apparatuses 10_1 to 10_n is fixed at a specific position, and may process information on the medical treatment result input from the doctor performing the medical treatment and to transmit it to an adjacent medical treatment apparatus and the central management apparatus 20. In addition, each of the plurality of medical treatment apparatuses 10_1 to 10_n may receive information on the service user from the vehicle 30, and may display the received information on the service user to an external display such that it may be considered for medical treatment.

The central management apparatus 20 may receive information on the medical treatment result and information on the service user from the plurality of medical treatment apparatuses 10_1 to 10_n, identify a progress situation of the drive-through medical treatment, and send the required information and control instruction to each of the plurality of medical treatment apparatuses 10_1 to 10_n such that medical treatment required for the service user may be performed based on identified situation. At this time, the central management apparatus 20 may determine and update communication protocol and data format for sending and receiving data between the vehicle 30 and the plurality of medical treatment apparatuses 10_1 to 10_n according to the vehicle 30, and may notify the same to the plurality of medical treatment apparatuses 10_1 to 10_n.

The communication scheme between the plurality of medical treatment apparatuses 10_1 to 10_n is not limited to WLAN, and the communication scheme between vehicle 30 and the plurality of medical treatment apparatuses 10_1 to 10_n is not limited to WPAN. Any one of various communication schemes may be applied to a drive-through medical treatment system 1 according to an exemplary embodiment.

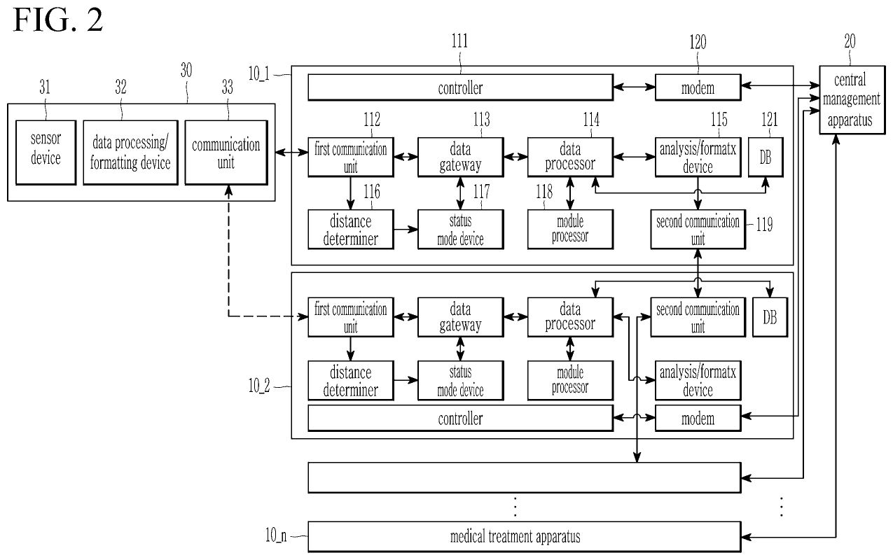

FIG. 2 illustrates an exemplary configuration of a vehicle and a medical treatment apparatus according to an exemplary embodiment.

As shown in FIG. 2, the vehicle 30 includes a sensor device 31, a data processing/formatting device 32, and a communication unit 33.

The sensor device 31 may include various sensors that may identify a current status of the service user in the vehicle 30. For example, the sensor device 31 may include a camera that may photograph an image of the service user, a heart rate sensor that may detect a heart rate of the service user, a temperature sensor that may measure a body temperature of the service user, a sensor that may record a voice of the service user.

The data processing/formatting device 32 may collect and process information on the service user to generate the service user information, and may determine data format suitable for the generated service user information.

The data processing/formatting device 32 may process information on the service user obtained through the sensor device 31 without separate analysis. For example, the data processing/formatting device 32 may process information on the service user that includes an image photographing a face of the service user, a body temperature and heart rate of the service user during a predetermined monitoring period, and voice data of the service user, according to a predetermined data format.

Alternatively, the data processing/formatting device 32 may analyze information on the service user obtained through the sensor device 31 and include it in the service user information. For example, the data processing/formatting device 32 may extract a maximum heart rate of the service user during the monitoring period, extract a maximum level of voice of the service user, and extract a maximum temperature from body temperatures. Furthermore, the data processing/formatting device 32 may estimate at least one of a health status and an emotional status of the service user based on the photographed image of the service user. In this case, the data processing/formatting device 32 may process the service user information in a text data format that includes at least one of a health status and an emotional status of the service user, maximum heart rate, maximum voice, and maximum body temperature, instead of the predetermined data format.

In addition, the data processing/formatting device 32 may process the service user information in a JSON format when the capacity of the service user information exceeds a predetermined threshold value.

The communication unit 33 may transmit the service user information generated by the data processing/formatting device 32 to the plurality of medical treatment apparatuses 10_1 to 10_n in a WPAN scheme.

FIG. 2 illustrates in detail two medical treatment apparatuses 10_1, and 10_2 among the plurality of medical treatment apparatuses 10_1 to 10_n. In an exemplary embodiment, the plurality of medical treatment apparatuses 10_1 to 10_n are described to have the same configuration. However, the present disclosure is not limited thereto, and if necessary, another component may be added, or unnecessary components may be excluded.

The medical treatment apparatus 10_1 includes a controller 111, a first communication unit 112, a data gateway 113, a data processor 114, an analysis/format device 115, a distance determiner 116, a status mode device 117, and a module processor 118.

The controller 111 may monitor an operation performed by the medical treatment apparatus and control each component according to a monitoring result. For example, when communication protocol is different between the vehicle 30 and the medical treatment apparatus 10_1 to 10_n, or when data format of the service user information received from the vehicle 30 is different from the predetermined protocol or data format of the medical treatment apparatus 10_1, the controller 111 notifies it to the central management apparatus 20 through a modem 120, and requests information for setting protocol and data format. When the information on protocol and data format is received through the modem 120, the controller 111 delivers the received information to the analysis/format device 115.

The first communication unit 112 may request information required for the medical treatment service to the vehicle 30 or receive it from the vehicle 30, through communication in the WPAN scheme with the communication unit 33 of the vehicle 30. Bluetooth Low Energy (BLE), which is one of the WPAN schemes may be applied as the communication scheme between the vehicle 30 and the plurality of medical treatment apparatuses 10_1 to 10_n in an exemplary embodiment.

The data gateway 113 delivers the data received from the first communication unit 112 to the data processor 114 and transmits data received from the data processor 114 to the first communication unit 112, to perform data communication between the vehicle and the medical treatment apparatus 10_1.

The data processor 114 processes the data received from the data gateway 113 according to the data format selected by the analysis/format device 115 to generate medical treatment data. The data processor 114 may transmit the medical treatment data to the module processor 118.

The analysis/format device 115 selects a data format by analyzing data received from the data gateway 113. The analysis/format device 115 may select a protocol and format that may be used for data processing from a protocol and format table based on a result of analyzing the received data, and inform the data processor 114 of the selected data format. The analysis/format device 115 may notify to the controller 111 that there is no protocol and format suitable for the data received from the vehicle 30 if there is no protocol and format that may be used for data processing in the protocol and format table. In this case, the controller 111 may request information on protocol and format to the central management apparatus 20 through the modem 120. In response to the request, the central management apparatus 20 may transmit information on changed protocol and format to the modem 120. The controller 111 delivers the information on new protocol and format received through the modem to the analysis/format device 115. The analysis/format device 115 adds the delivered protocol and format to the protocol and format table, selects a data format, and transmits it to the data processor 114.

The distance determiner 116 may measure a strength of signal received from the vehicle 30, and based on the received signal strength indication (RSSI), may estimate a distance between the vehicle 30 and the medical shelter (for example, the medical shelter where the medical treatment apparatus 10_1 is positioned). When the estimated distance is within a predetermined reference distance, the distance determiner 116 may determine that the vehicle 30 has entered a corresponding medical shelter and generate an Enable signal.

Upon receiving the Enable signal from the distance determiner 116, the status mode device 117 enables the data gateway 113 and the module processor 118, synchronously with the Enable signal.

The module processor 118 performs information processing required for performing the medical treatment through the medical treatment apparatus 10_1 based on the medical treatment data received from the data processor 114. For example, the module processor 118 processes information required for performing various operation related to the medical treatment service, such as receiving the medical treatment result, processing and displays the received medical treatment result, receiving information from the service user or the doctor, and displaying required information to the service user or the doctor. The module processor 118 may be provided in each of the medical treatment apparatuses, and may process information required according to medical treatment or work performed by the medical treatment apparatuses.

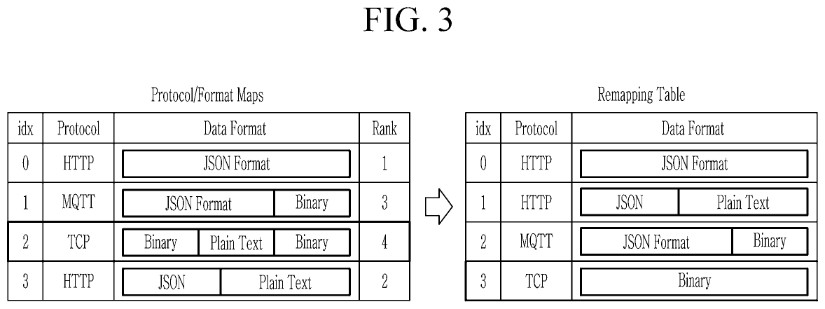

FIG. 3 illustrates determining protocol and format according to an exemplary embodiment.

As shown in FIG. 3, the central management apparatus 20 manages the protocol and format map as a pair composed of protocol and data format. In the left table of FIG. 3, “idx” indicates an index number, “protocol” indicates a communication protocol scheme between the vehicle 30 and the plurality of medical treatment apparatuses 10_1 to 10_n, “Data format” indicates a format of data sent and received through communication between the vehicle 30 and the plurality of medical treatment apparatuses 10_1 to 10_n, and “Rank” indicates ranking based on a statistical usage rate by which format pair and protocol corresponding to each index number is used and a sending and receiving success rate.

The central management apparatus 20 includes a client 21, an analysis determiner 22, a reconstruction device 23, and a remapping device 24, as shown in FIG. 1.

The client 21 may collect information on protocol and format from the plurality of medical treatment apparatuses 10_1 to 10_n, and may transmit information on updated protocol and format to the plurality of medical treatment apparatuses 10_1 to 10_n. The information on protocol and format from the plurality of medical treatment apparatuses 10_1 to 10_n may include, a request for a new protocol and format received from the medical treatment apparatus, information on whether the current protocol and format is used, information on success/failure of transmission performed through the current protocol and format, and the like.

The analysis determiner 22 analyzes the information on protocol and format collected by the client 21, and thereby, as shown in the left table in FIG. 3, calculates a usage rate and a data transmission success rate with respect to each pair within the protocol and format group, and sets ranking for each pair according to the usage rate and the data transmission success rate. The analysis determiner 22 may set a ranking for each pair in descending order from a highest value based on a sum value of values obtained by multiplying each of the usage rate and the success rate by a reflection ratio.

The analysis determiner 22 may make a discard decision with respect to a pair below a certain level, for example, when each of the usage rate and the success rate is below a predetermined threshold value, when a sum value of values obtained by multiplying each of the usage rate and the success rate by a reflection ratio is below a predetermined threshold value. The analysis determiner 22 may transmit the discard decision to the reconstruction device 23.

When a discarded pair exists in the protocol and format group, the reconstruction device 23 may generate a new protocol and format pair. The reconstruction device 23 may arbitrarily reconstruct a new data format. For example, when a protocol and format pair whose “idx” is 2 in the left table of FIG. 3 is discarded, the data format may be reconstructed as a protocol and format pair whose “idx” is 3 in the right table of FIG. 3.

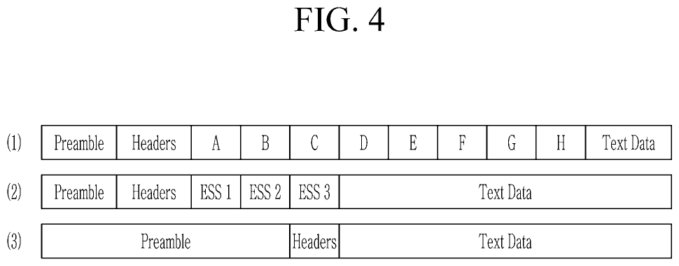

FIG. 4 illustrates an example of reconstructing a data format according to an exemplary embodiment.

The reconstruction device 23 may reconstruct the data format in various schemes other than the data format shown in FIG. 3.

As shown in FIG. 4, the data format may be implemented in the order of Preamble, Header, and Data. As shown in row (1) of FIG. 4, various data may be positioned in a plurality of format spaces A to H positioned between the Header and the Text Data. In addition, as shown in row (2) of FIG. 4, extended service sets (ESS) ESS1 to ESS3 may be positioned between the Header and the Text Data.

The reconstruction device 23 transmits a new protocol and format pair including a new data format to the remapping device 24.

The remapping device 24 excludes the protocol and format pair discarded by the analysis determiner 22, and includes new protocol and format pair generated by the reconstruction device 23, to perform remapping with respect to the protocol and format group. Then, as shown in the right table of FIG. 3, a new protocol and format pair (TCP+Binary) of “idx” 3 is added, and the existing protocol and format pair (TCP+(Binary+Plain Text+Binary)) of “idx” 2 is deleted.

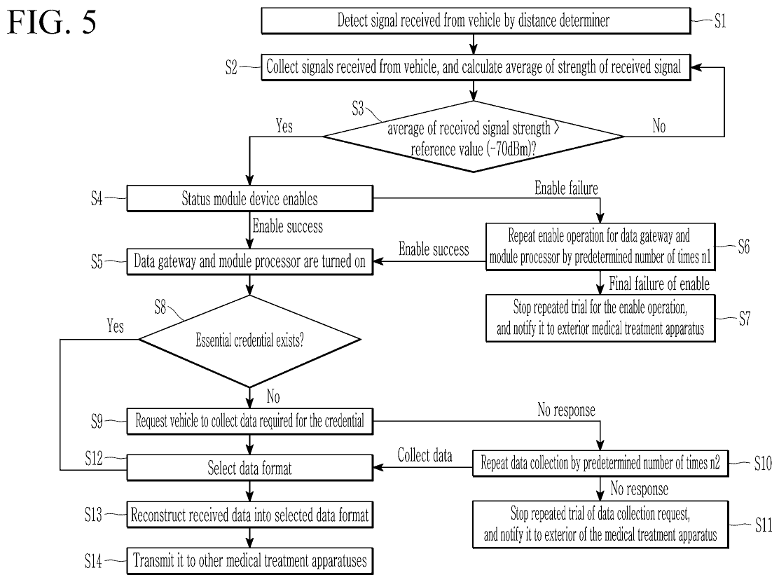

FIG. 5 is a flowchart showing a drive-through medical treatment method according to an exemplary embodiment.

First, at step S1, the vehicle 30 approaches the drive-through medical treatment place, and a signal from the vehicle 30 is detected by the distance determiner 116.

At step S2, the distance determiner 116 collects signals received from the vehicle 30, and calculates an average of strength of the received signal.

At step S3, the distance determiner 116 determines whether the calculated average of the received signal strength is larger than a predetermined reference value (e.g., −70 dBm).

When the average of the received signal strength is larger than the reference value (S3-Yes), the distance determiner 116 generates an Enable signal and transmits it to the status mode device 117, and thereby the status mode device 117 is enabled, at step S4. When the average of the received signal strength is less than or equal to the reference value, the step S2 is repeated.

At step S5, the status mode device 117 enables the data gateway 113 and the module processor 118, and when the data gateway 113 and the module processor 118 are enabled thereby (enable success), the data gateway 113 and the module processor 118 are turned on. Alternatively, the data gateway 113 and the module processor 118 may not be enabled (enable failure).

At step S6, when the data gateway 113 and the module processor 118 are not enabled, the status mode device 117 repeats the enable operation for the data gateway 113 and the module processor 118 by a predetermined number of times n1.

At step S7, when the data gateway 113 and the module processor 118 are not enabled to be turned on even if the status mode device 117 repeats the enable operation by the predetermined number of times (final failure of enable), repeated trial for the enable operation is stopped, and the status that the data gateway 113 and the module processor 118 are not turned on is notified to an exterior of the medical treatment apparatus 10_1 such that the doctor may physically contact the service user.

At step S8, the data gateway 113 is turned on, and determines whether the data received from the vehicle 30 has an essential credential.

At step S9, when the essential credential does not exist in the data received from the vehicle 30, the data gateway 113 requests the vehicle 30 to collect data required for the credential through the first communication unit 112.

When the essential credential exists in the data received from the vehicle 30 (S8-Yes) or when the data required for the credential is collected from the vehicle 30 in response to the request of the step S9, a subsequent step S12 is performed.

At this time, when a response with respect to the request of the step S9 is not received, the data gateway 113 repeats to request to collect the data by a predetermined number of times n2, at step S10. When the data in response to the data collection request are collected within the predetermined number of times, the subsequent step S12 is performed. Otherwise, at step S11, repeated trial of data collection request is stopped, and the status that the essential credential is not made is notified to an exterior of the medical treatment apparatus 10_1 such that the doctor may physically contact the service user.

At step S12, the analysis/format device 115 receives data from the data gateway 113, and analyzes the received data to select a corresponding data format in the protocol and format table.

At step S13, the data processor 114 receives the data from the data gateway 113, and reconstruct the received data into the data format selected by the analysis/format device 115.

At step S14, the data processor 114 may transmit the reconstructed data to another medical treatment apparatus 10_2 through a second communication unit 119.

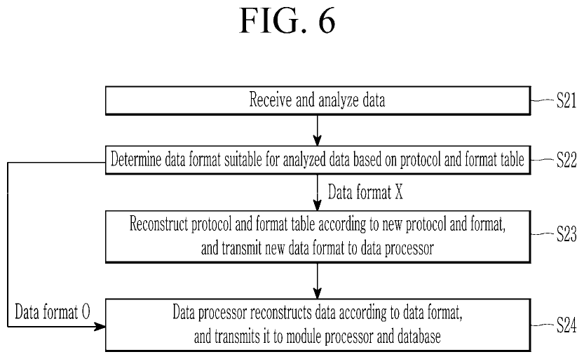

FIG. 6 illustrates a data format determining method according to an exemplary embodiment.

First, at step S21, the medical treatment apparatus 10_1 receives data from the vehicle 30, and the analysis/format device 115 receives and analyzes the data.

At step S22, the analysis/format device 115 determines a data format suitable for the analyzed data, based on the protocol and format table. When the suitable data format exists (data format O), the analysis/format device 115 delivers the determined data format to the data processor 114.

When the suitable data format does not exists (data format X), at step S23, the analysis/format device 115 reconstructs the protocol and format table according to new protocol and format received from the central management apparatus 20, and transmits new data format to the data processor 114. At this time, index numbers of the protocol and format table may be rearranged.

At step S24, the data processor 114 may reconstruct the data according to the data format, and may transmit it to the module processor 118 and database (DB) 121.

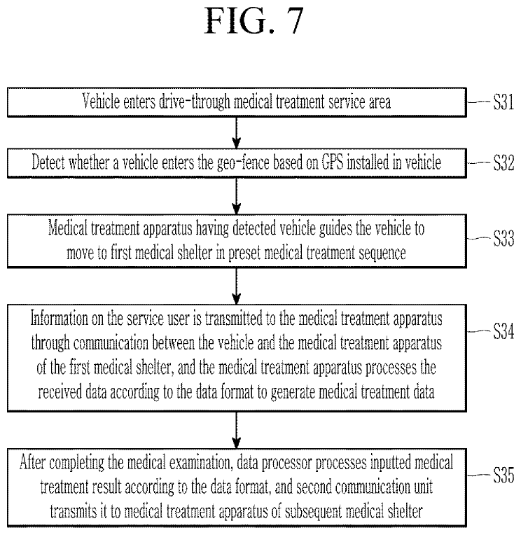

FIG. 7 illustrates an example applied with a drive-through medical treatment system according to an exemplary embodiment.

First, at step S31, the vehicle 30 enters a drive-through medical treatment service area.

At step S32, the drive-through medical treatment system 1 may install a virtual fence ‘geo-fence’ within a predetermined range of a hospital, and may detect whether the vehicle 30 enters the geo-fence through a positioning technique using the GPS installed in the vehicle 30. Here, the term hospital means a hospital installed with the drive-through medical treatment place. When the vehicle 30 enters the geo-fence, the drive-through medical treatment system 1 may notify it to the management server of the hospital and the plurality of medical treatment apparatuses 10_1 to 10_n provided in the plurality of medical shelters. The plurality of medical treatment apparatuses 10_1 to 10_n may start a search whether the vehicle 30 is detected by using the distance determiner 116.

While the vehicle 30 is moving, the vehicle 30 may be first detected by at least one of the plurality of medical treatment apparatuses 10_1 to 10_n. At step S33, the medical treatment apparatus having detected the vehicle 30 guides the vehicle 30 to move to a first medical shelter in a preset medical treatment sequence. In addition, when the vehicle 30 may communicate with all of the plurality of medical treatment apparatuses 10_1 to 10_n, the plurality of medical treatment apparatuses 10_1 to 10_n may detect that the vehicle 30 has entered the service area. In addition, the medical treatment apparatus having detected the vehicle 30 may notify it to other medical treatment apparatuses through WiFi communication. The communication between the vehicle 30 and the plurality of medical treatment apparatuses 10_1 to 10_n may be processed by BLE communication.

At step S34, information on the service user is transmitted to the medical treatment apparatus 10_1 through communication between the vehicle 30 and the medical treatment apparatus 10_1 of the first medical shelter, and the medical treatment apparatus 10_1 processes the received data according to the data format to generate the medical treatment data. At this time, the data received from the vehicle 30 may include, gender, age, emotion (Vibe), temperature, heart rate, blood pressure, body size, eye color, hair color, voice, and the like of the service user, and may also include running information of the vehicle 30. The medical treatment apparatus 10_1 may notify the doctor that the service user has arrived or may display it on a monitor provided in the medical treatment apparatus 10_1.

The doctor may perform a medical examination and, after completing the medical examination, input the medical treatment result through the user interface provided on the medical treatment apparatus 10_1. At step S35, the module processor 118 transmits the inputted medical treatment result to the data processor 114, the data processor 114 processes the medical treatment result according to the data format to generate the medical treatment data, and the second communication unit 119 receives the processed medical treatment data from the data processor 114 and transmits it to the medical treatment apparatus 10_2 of a subsequent medical shelter. At this time, the controller 111 may transmit an instruction to move to a subsequent medical shelter to the vehicle 30 through the first communication unit 112, and may notify through the second communication unit 119 that the service user is moving to the subsequent medical shelter.

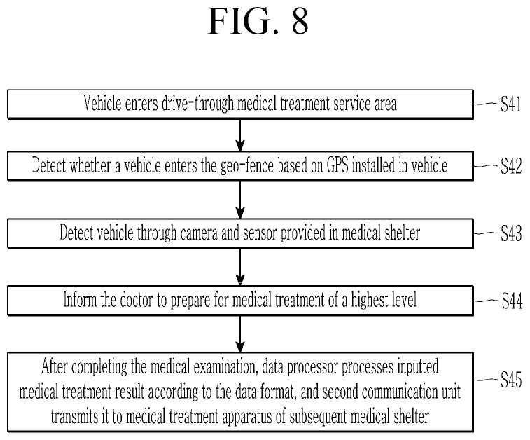

FIG. 8 illustrates another example applied with a drive-through medical treatment system according to an exemplary embodiment.

First, at step S41, the vehicle 30 enters the drive-through medical treatment service area.

At step S42, the drive-through medical treatment system 1 may install a virtual fence ‘geo-fence’ within a predetermined range of a hospital, and may detect whether the vehicle 30 enters the geo-fence through a positioning technique using the GPS installed in the vehicle 30. Here, the term hospital means a hospital installed with the drive-through medical treatment place. When the vehicle 30 enters the geo-fence, the drive-through medical treatment system 1 may notify it to the management server of the hospital and the plurality of medical treatment apparatuses 10_1 to 10_n provided in the plurality of medical shelters. The plurality of medical treatment apparatuses 10_1 to 10_n may start a search whether the vehicle 30 is detected by using the distance determiner 116.

At this time, wireless communication between the vehicle 30 and the plurality of medical treatment apparatuses 10_1 to 10_n may be impossible due to wireless communication system of the vehicle 30, for example, when the BLE is not provided or malfunctioning.

Then, at step S43, the vehicle 30 may be detected through a camera and a sensor provided in the medical shelter. The plurality of medical treatment apparatuses 10_1 to 10_n prepares a unidirectional service.

At step S44, when the entrance of the vehicle 30 is detected through the camera and sensor provided in the medical shelter, the plurality of medical treatment apparatuses 10_1 to 10_n may inform the doctor to prepare for medical treatment of a highest level.

The doctor may perform a medical examination and, after completing the medical examination, input the medical treatment result through the user interface provided on the medical treatment apparatus 10_1. At step S45, the module processor 118 transmits the inputted medical treatment result to the data processor 114, the data processor 114 processes it according to the data format, the second communication unit 119 receives the processed medical treatment data from the data processor 114 and transmits it to the medical treatment apparatus 10_2 of the subsequent medical shelter. At this time, the controller 111 may transmit an instruction to move to a subsequent medical shelter to the vehicle 30 through the first communication unit 112, and may notify through the second communication unit 119 that the service user is moving to the subsequent medical shelter.

While this disclosure has been described in connection with what is presently considered to be practical exemplary embodiments, it is to be understood that the disclosure is not limited to the disclosed embodiments. On the contrary, it is intended to cover various modifications and equivalent arrangements included within the spirit and scope of the appended claims.

1. A drive-through medical treatment system utilizing a vehicle, comprising:
a plurality of medical treatment apparatuses provided in a plurality of medical shelters; and
a central management apparatus configured to receive information on a medical treatment result, and to receive information on a service user from the plurality of medical treatment apparatuses to identify a progress situation of the drive-through medical treatment, and to send required information and a control instruction to each of the plurality of medical treatment apparatuses such that medical treatment required for the service user may be performed based on an identified situation;
wherein at least one of the plurality of medical treatment apparatuses is configured to receive information on the service user to generate medical treatment data, to process information on a medical treatment result of the service user, and to transmit the processed information to an adjacent medical treatment apparatus and the central management apparatus.

2. The drive-through medical treatment system of claim 1, wherein the vehicle comprises:
a sensor device configured to check a current status of the service user;
a data processing or formatting device configured to process information on the service user obtained through the sensor device according to data format to generate service user information; and
a communication unit configured to transmit the service user information to the plurality of medical treatment apparatuses.

3. The drive-through medical treatment system of claim 2, wherein the information on the service user includes an image photographing a face of the service user, a body temperature of the service user, a heart rate of the service user during a predetermined monitoring period, and voice data of the service user.

4. The drive-through medical treatment system of claim 2, wherein the data processing or formatting device is configured to analyze the information on the service user, to extract a maximum heart rate of the service user during a predetermined monitoring period, to extract a maximum level of voice of the service user, to extract a maximum temperature from measured body temperatures of the service user, and to estimate at least one of a health status and an emotional status of the service user based on a photographed image of the service user, and wherein the data processor or formatting device is further configured to process the analyzed information in a text data format to generate the service user information.

5. The drive-through medical treatment system of claim 1, wherein each of the plurality of medical treatment apparatuses comprises:
a controller configured to monitor operations performed by each medical treatment apparatus and to perform a control operation according to a monitoring result;
a first communication unit performing communication with the vehicle;
a data gateway configured to receive data from the first communication unit or to transmit data to the first communication unit;
an analysis or format device configured to analyze data received from the data gateway to select a data format; and
a data processor configured to process the data received from the data gateway according to the data format selected by the analysis or format device to generate medical treatment data.

6. The drive-through medical treatment system of claim 5, further comprising:
a module processor configured to receive the medical treatment data from the data processor, and to perform processing of information required for performing medical treatment based on the medical treatment data.

7. The drive-through medical treatment system of claim 5, wherein the analysis or format device is configured to select a protocol and format that may be used for data processing from a protocol and format table based on a result of analyzing the received data, and to inform the selected data format to the data processor.

8. The drive-through medical treatment system of claim 5, wherein:
the analysis or format device is configured to, when a protocol and format that may be used for data processing does not exists in a protocol and format table, to the controller that the protocol and format suitable for the data received from the vehicle does not exist; and
the controller is configured to request the information on protocol and format from the central management apparatus.

9. The drive-through medical treatment system of claim 8, wherein:
the controller is configured to receive information on changed protocol and format from the central management apparatus, and to deliver the received information to the analysis or format device; and
the analysis or format device is configured to add the delivered protocol and format to the protocol and format table, and to select the added data format to transmit the selected data format to the data processor.

10. The drive-through medical treatment system of claim 6, wherein each of the plurality of medical treatment apparatuses further comprises:
a distance determiner configured to measure a strength of a signal received from the vehicle, and to estimate a distance between the vehicle and a medical shelter based on the measured received signal strength indication (RSSI).

11. The drive-through medical treatment system of claim 10, further comprising:
a status mode device configured to, when an enable signal is generated due to determining by the distance determiner that the vehicle has entered a corresponding medical shelter, enable the data gateway and the module processor synchronously with the enable signal.

12. The drive-through medical treatment system of claim 1, wherein the central management apparatus comprises:
a client configured to collect information on protocol and format from the plurality of medical treatment apparatuses, and to transmit information on updated protocol and format to the plurality of medical treatment apparatuses; and
an analysis determiner configured to analyze the information on protocol and format collected by the client to calculate a usage rate and a data transmission success rate with respect to each pair within the protocol and format group, and to set ranking for each pair according to the usage rate and the data transmission success rate.

13. The drive-through medical treatment system of claim 12, wherein the analysis determiner is configured to, when each of the usage rate and the success rate is below a predetermined threshold value, or when a sum value of values obtained by multiplying each of the usage rate and the success rate by a reflection ratio is below a predetermined threshold value, decide to discard a corresponding pair within the protocol and format group decision.

14. The drive-through medical treatment system of claim 13, wherein the central management apparatus further comprises a reconstruction device configured to generate a new protocol and format pair when a discarded pair exists in the protocol and format group.

15. The drive-through medical treatment system of claim 14, wherein the central management apparatus further comprises:
a remapping device performing remapping with respect to the protocol and format group by excluding the protocol and format pair discarded by the analysis determiner, and by including the new protocol and format pair generated by the reconstruction device.

16. A drive-through medical treatment method for receiving a drive-through medical treatment service by using a vehicle, comprising:
detecting, by a distance determiner, a signal from the vehicle;
collecting, by the distance determiner, signals received from the vehicle, and calculating an average of strength of the signal;
determining, by the distance determiner, whether the calculated average of signal strength is larger than a predetermined reference value;
enabling, by the distance determiner, a status mode device, when the average of the signal strength is larger than the reference value;
enabling, by the status mode device, a data gateway to be turned on;
determining, by the turned-on data gateway, whether the data received from the vehicle has an essential credential; and
analyzing, by an analysis or format device, the data delivered from the data gateway to select a corresponding data format in a protocol and format table, when the received data has the essential credential.

17. The drive-through medical treatment method of claim 16, further comprising:
repeating, by the status mode device, an enable operation with respect to the data gateway by a predetermined number of times repeat, when the data gateway is not enabled by the status mode device.

18. The drive-through medical treatment method of claim 16, further comprising:
requesting the vehicle, by the data gateway, to collect data required for the credential, when the essential credential does not exist in the data received from the vehicle; and
repeating, by the data gateway, data collect by a predetermined number of times, when a response with respect to the collection request is not received.

19. A drive-through medical treatment method, comprising:
analyzing, by an analysis or format device, data received from a vehicle;
determining, by the analysis or format device, a suitable data format for the analyzed data, based on a protocol and format table;
reconstructing, when the suitable data format does not exists, by the analysis or format device, the protocol and format table according to a new protocol and format received from central management apparatus, and transmitting the new data format to a data processor; and
reconstructing, by the data processor, the data received from the vehicle according to the data format.

20. A drive-through medical treatment method, comprising:
detecting, by the drive-through medical treatment system, by using a geo-fence, a vehicle entering a drive-through medical treatment service area;
informing a plurality of medical treatment apparatuses, by the drive-through medical treatment system, when the vehicle enters the geo-fence;
detecting, by a distance determiner of at least one of the plurality of medical treatment apparatuses, the vehicle, and guiding the vehicle to move to a first medical shelter in a preset medical treatment sequence;
transmitting information on a service user to the medical treatment apparatus through communication between the vehicle and the medical treatment apparatus of the first medical shelter;
processing, by the medical treatment apparatus, received data according to a data format to generate medical treatment data; and
processing an inputted medical treatment result according to the data format to generate medical treatment data after completing a medical examination, and transmitting the generated medical treatment data to a medical treatment apparatus of a subsequent shelter among the plurality of medical treatment apparatuses.
